(19) United States
(12) Patent Application Publication
(10) Pub. No.: US 2014/0303739 A1
(43) Pub. Date: Oct. 9, 2014

(54) ORTHOPAEDIC BEARING AND METHOD OF ASSESSING AN ORTHOPAEDIC IMPLANT

(71) Applicant: Isis Innovation Limited, Oxford (GB)

(72) Inventors: Michael Mentink, Oxford (GB); David Murray, Oxford (GB); Harinderjit Singh Gill, Bath (GB); Bernard Kendrik Van Duren, Oxford (GB)

(73) Assignee: ISIS INNOVATION LIMITED, Oxford (GB)

(21) Appl. No.: 14/365,595

(22) PCT Filed: Dec. 14, 2012
(86) PCT No.: PCT/GB2012/053150

§ 371 Date: Jun. 13, 2014

(30) Foreign Application Priority Data
Dec. 14, 2011 (GB) 1121518.3

(51) Int. Cl. A61F 2/38

(52) U.S. Cl. CPC A61F 2/3836; A61F 2/3886; A61F 2/389
USPC 623/20.27

(57) ABSTRACT

There is provided an orthopaedic bearing comprising at least one capacitive sensing element arranged within the bearing material, and operable to measure a change in capacitance resultant from any compression or tension of the bearing during use. There is also provided a method of assessing an orthopaedic implant including an instrumented orthopaedic bearing.

### FIELD OF THE INVENTION

This invention relates to orthopaedic implants in general, and to an apparatus and method for assessing one or more parameters of an orthopaedic implant during use.

### BACKGROUND OF THE INVENTION

Orthopaedic implants (i.e. replacement joints) are typically used to replace missing, worn out, diseased or otherwise reduced function joints, such as knees, shoulders, elbows, hips and the like. A typical cause of reduced function in joints is, for example, osteoarthritis, which is a joint disease in which cartilage becomes irreversibly damaged. Arthritis results in painful joints and therefore can have an adverse effect on movement patterns and activities of daily life. For example, in persons with an arthritic knee, this can result in restricted motion and severely reduced quality of life.

The aetiology of osteoarthritis is in some cases disputed; the difference between people may be physiological and/or mechanical, with predisposing factors being age, obesity, occupation and trauma. From a mechanical viewpoint, the cause of osteoarthritis is believed to be a difference in mechanical loading of the knee in healthy individuals compared to individuals that have a high risk of developing osteoarthritis.

To obtain a better understanding of the forces acting on orthopaedic implants used in knee replacements in particular, attempts have been made to estimate the mechanical force and moment from external gait measurements, mechanical simulations (in vitro assessment), mechanical computer simulations (i.e. in silico assessment) and telemetered implants. However, all these methods have shortcomings; force estimates from external gait measurements are not very accurate. The accuracy of simulations is dependent on the data provided by measurements, for instance from telemetered implants. Knee kinematics in vitro and measured kinematics in vivo are different; whereby forces in vivo are reported lower than in vitro. Therefore, in vitro measurements cannot provide an accurate picture of forces in vivo, and accurate measurements in vivo are necessary.

Accordingly, there is a desire to provide an improved orthopaedic implant that is capable of providing more accurate measurements of the forces experienced by the orthopaedic implant in vivo. This data can then be used to improve gait models, orthopaedic implant models, orthopaedic implant procedures, orthopaedic implants, and gain a better understanding of joint loading.

### SUMMARY OF THE INVENTION

According to the invention there is provided an orthopaedic bearing suitable for use with an orthopaedic implant, the orthopaedic bearing comprising at least one deformable portion, the at least one deformable portion being formed from a flexible bearing material adapted to deform under load, characterised in that at least one capacitive sensing element is embedded within the deformable portion, the capacitive sensing element being operable to measure a change in capacitance resultant from a deformation of the deformable portion.

In this way, the deformation of the orthopaedic bearing during use causes a change in the distance between capacitive elements, which in turns causes a measurable change in capacitance. As such, by measuring the change in capacitance, a measurement of the forces being applied to the orthopaedic bearing can be obtained. Such information is very useful to those designing orthopaedic implants.

Optionally, the orthopaedic bearing comprises a plurality of capacitive sensing elements arranged within the at least one deformable portion of the orthopaedic bearing and operable to measure changes in capacitance over an area of the bearing resultant from a deformation of the deformable portion of the orthopaedic bearing during use. By using many capacitive sensing elements, we can measure forces over an area, and are not limited to measurements at one particular spot.

Optionally, the plurality of capacitive sensing elements comprises an array, and wherein the array is formed as a grid in a regular or irregular pattern.

Optionally, the plurality of capacitive sensing elements are distributed in three dimensions, length, width and height, of the orthopaedic bearing. In this way forces throughout the bearing may be measured.

Optionally, the array is a square or circular pattern, and a centre of the square or circular pattern is at an estimated Centre Of Pressure (COP). The COP is of particular interest in examining wear in an orthopaedic implant.

Optionally, the orthopaedic bearing further comprises an accelerometer operable to provide acceleration data for the orthopaedic bearing during use, in order to correlate position of knee with capacitance changes in at least one capacitive sensing element, or to correlate acceleration of the knee with capacitive changes in at least one capacitive sensing element.

Optionally, the orthopaedic bearing further comprises a temperature sensor operable to measure a temperature of the orthopaedic bearing during use, said temperature measurement being used to compensate for variation in the measured capacitance due to temperature change.

Optionally, the orthopaedic bearing further comprises a plurality of temperature sensors, operable to determine point temperatures of the bearing material across an area of the orthopaedic bearing, wherein the points correspond to capacitive sensing element locations.

Optionally, the orthopaedic bearing further comprises electronic circuitry encapsulated within a portion of the orthopaedic bearing that does not experience compression during use, operable to convert capacitance changes in the at least one capacitive sensing element into data for sending out over a communications link.

Optionally, the at least one capacitive sensing element is connected to at least one lead that follows an undulating path. In this way, the leads may be connected to further electronic circuitry, such as an induction coil for transmitting data. The orthopaedic bearing is formed using compression moulding wherein the capacitive sensing elements are placed in the mould before the compression stage, so as to embed them within the bearing material. The use of undulating leads allows the leads to stretch without breaking during the moulding process, so that they remain connected to the capacitive sensing elements.

Optionally, the leads undulate at approximately 60°. This provides effective stretching of the leads during the compression moulding process.

Optionally, the orthopaedic bearing further comprises one or more locating legs adapted to locate the at least one capacitive sensing element correctly within the orthopaedic bearing. The legs allow the capacitive sensing elements to be positioned more accurately within the orthopaedic bearing, by limiting the movement of the capacitive sensing elements during the compression moulding process.

Optionally, the one or more locating legs project from the corners of a module comprising the at least one capacitive sensing element. This provides for good positioning.

Optionally, the module is substantially rectangular in shape and the one or more locating legs project at an angle that is substantially 45° to each adjacent side.

Optionally, the one or more locating legs have a bulbous distal end. Optionally, the one or more locating legs are angled. By adjusting the shape, arrangement and terminations of the locating legs of the at least one capacitive sensing element, improved positioning of the capacitive sensing element within the orthopaedic bearing can be provided for. For example, an orthopaedic bearing for use in a UKR is not symmetrical in any direction, resulting in a set of locating legs where each leg is slightly different from the others.

Optionally, the deformation of the deformable portion comprises compression thereof. Additionally, the deformation of the deformable portion may comprise tension thereof. Each is possible within the bearing and each provides a change in distance between capacitive sensing elements thus a change in capacitance and thus an indication of forces.

Optionally, the at least one capacitive sensing element comprises a pair of parallel overlapping plates. In this way, the capacitive sensing element acts as a standard parallel plate capacitor.

Optionally, plurality of capacitive sensing elements are formed by a grid of overlapping conductors, said overlapping conductors comprising a first plurality of parallel and spaced apart conductors at a first orientation and a second plurality of parallel and spaced apart conductors at a second orientation, wherein a capacitive sensing element is formed at an overlapping junction between each one of the first and second plurality of parallel and spaced apart conductors. This is a particularly convenient manner of forming an array of parallel plate capacitors. Preferably, the first orientation is orthogonal to the second orientation, providing rectangular capacitors.

Optionally, at least one capacitive sensing element is formed from two parallel plates adjacent to one another in use, wherein compression of the orthopaedic bearing causes the parallel plates to move towards each other and tension of the orthopaedic bearing causes the parallel plates to move away from one another.

Optionally, the orthopaedic bearing comprises at least one capacitive sensing element arranged within the bearing material, and operable to measure a change in capacitance resultant from any change in distance between a metallic bone insert and the capacitive sensing element within the orthopaedic bearing during use.

Optionally, the orthopaedic bearing comprises a plurality of capacitive sensing elements arranged within the at least one deformable portion of the orthopaedic bearing, wherein the plurality of capacitive sensing elements are positioned either in a plane parallel to the bottom of the bearing in use, or in a plane perpendicular to the bottom of the bearing in use, or a combination of either plane orientations

Optionally, at least one capacitive sensing element is formed from two adjacent co-planar plates operable to measure capacitance as a function of a proximity of the adjacent plates to a metallic bone insert impinging on the orthopaedic bearing.

Optionally, the orthopaedic bearing is used with a tibial tray including a peg, and the orthopaedic bearing further comprises capacitive sensing elements arranged to measure a change in capacitance resultant from any compression or tension of the bearing surrounding the peg during use.

Optionally, orthopaedic bearing is used with a tibial tray and comprises a post that articulates with a femoral cam, and the orthopaedic bearing further comprises capacitive sensing elements arranged to measure a change in capacitance resultant from any compression or tension of the bearing caused by the articulating femoral cam during use.

Optionally, the plurality of capacitive sensing elements are positioned either in a plane parallel to the bottom of the bearing in use, or in a plane perpendicular to the bottom of the bearing in use, or a combination of both planes.

There is also provided an orthopaedic implant comprising the orthopaedic bearing as herein described.

There is also provided a method of assessing an orthopaedic implant, comprising providing at least one orthopaedic bearing as described herein and determining, with the at least one orthopaedic bearing, at least one parameter of the orthopaedic implant during use.

Optionally, the method further comprises using a combined mathematical and Finite Element model, run on a computer during post-processing, to translate a measured change in capacitance into information indicative of any one or more of: deformation of the orthopaedic bearing; forces acting upon the orthopaedic bearing; wear of the orthopaedic bearing; or distance of metallic bone insert(s) in respect to the capacitive sensing element inside the bearing.

There is further provided a method wherein the combined mathematical and Finite Element model uses calibration data together with the measured change in capacitance to estimate any one or more of: compression or tension of a capacitive sensing element layer; a force that caused the compression or tension; and/or a Centre Of Pressure of the force that caused the compression or tension.

These and other aspects of the invention will be apparent from and elucidated with reference to the embodiments described hereinafter.

There is described an apparatus and method to measure one or more parameters of an orthopaedic implant during use, such as the level of deformation of the orthopaedic bearing within the implant, and the effect of the orthopaedic bearing and/or deformation of the orthopaedic bearing on the patient/user. Embodiments of the invention include an apparatus comprising an instrumented orthopaedic bearing having capacitive sensing elements formed within the orthopaedic bearing, for sensing/measuring the level of deformation of the bearing during use, preferably including the capability of locating particular compression measurements within the orthopaedic implant.

As described in more detail below, changes in capacitance are directly related to a change in distance between the plates (electrodes) of the capacitive sensing element, and hence can directly measure changes in compression on the orthopaedic implant. By mapping the measured compression levels across an orthopaedic implant/bearing during use, it is then possible to analyse important parameters of the orthopaedic implant, such as accurate or correct insertion or relative displacement of joint replacement components, effects on gait or joint mobility, wear of the bearing, and the like.

The orthopaedic bearing may be formed from any suitable material, such as polymers, and the like.

Optionally, the bearing material is deformable under pressure according to a known characteristic, preferably deformable under pressure with a known compression or tension characteristic.

An example of the present invention is described in terms of its application in a Uni-compartmental Knee Replacement (UKR) scenario (FIG. 1). However, it will be appreciated that embodiments of the present invention are not limited to this specific joint (i.e. knee) or form of orthopaedic implant (uni-compartmental). For example, the instrumented orthopaedic bearing may be used in any orthopaedic replacement situation, including and not limited to: Total Knee Replacement (TKR), Hip/shoulder/elbow replacement, prosthetic legs, prosthetic feet, prosthetic hands, and the like.

Within orthopaedic replacements, a bearing is a component, or part of a component, positioned between two other components within an orthopaedic implant, with the function of providing protection from damage when those two component articulate with each other directly. For example, a bearing may be the liner of the acetabulum component for a hip replacement

Bearings are made from a material that will allow for dampening of high frequency impulses or changes in load that otherwise would lead to wear of the two outer components, where the wear may include for example cracking, abrasive wear, denting, scarring, and other means of damage.

A bearing is designed in such a way that, even in the case of extensive wear, it can still perform its function within the orthopaedic implant, and so that the orthopaedic implant itself can perform its function, even though the bearing itself may be worn and/or abraded. One possible application of the present invention is to detect extensive wear, providing the option of preventative surgery to replace a worn bearing, or revise a sub-optimal implant, before complete failure. In the absence of early detection of such detrimental wear, it would be difficult to determine when such pre-emptive surgery should occur.

Regardless of the situation, an orthopaedic bearing is typically located between two or more fixed components involved in the joint articulation process. For example, in the UKR scenario, the orthopaedic bearing according to embodiments of the invention is located between a femoral component and a tibial tray component, and deforms under load. Deformation under load between the tibial and femoral components causes the orthopaedic bearing to be selectively compressed at certain points, which may be measured by apparatus according to embodiments of the present invention by using capacitive sensing elements located within the orthopaedic bearing.

Accordingly, by using a suitably-derived mathematical and/or Finite Element (FE) model of the replacement orthopaedic implant or joint, combined with the measured capacitance, the load on the replacement joint can be characterised and estimated. Furthermore, by measuring capacitance continuously, it can be determined how the load on the replacement orthopaedic implant or joint operates during a movement cycle, and important patient assessment information, such as a gait measurement, can also be characterised and estimated.

Equally, by taking static measurements using the orthopaedic bearing according to embodiments of the present invention, the distance of the femoral component and tibial tray with respect to the sensors can be estimated, which may have major applications in wear measurement of the replacement joint. By using multiple capacitive sensing elements within the orthopaedic bearing, the Centre of Pressure (COP), and pressure distribution of the load on a replacement joint during use can be measured, so that the relative movement of the femur and tibia can be more accurately determined, and thereby provide information on joint function during activity.

There are also described further embodiments of the present invention which use one or more integrated temperature sensors, so that the temperature data (either generally, in the case of a single temperature sensor, or locally mapped in the case of multiple temperature sensors being incorporated into the orthopaedic bearing) may be used in the modelling of the replacement joint (i.e. the model that links changes in capacitance compared to compression experience by the bearing), to increase the model's reliability, and hence accuracy of the instrumented orthopaedic bearing. Where a plurality of temperature sensors are used to determine the temperature of the bearing at multiple positions, they may be formed in a square, circular, hexagonal or any other suitably shaped grid, interspersed between the one or more capacitive sensing elements of the instrumented orthopaedic bearing. Preferably, the temperature sensors are located near to the capacitive sensing elements, in order to provide a more accurate measurement of the temperature being experienced by each capacitive sensing element, so that the computational model of each respective capacitive sensing element may be adjusted to provide more accurate capacitive sensing element readings.

Furthermore, embodiments may also include one or more accelerometers, which may be used to accurately determine the angle of the bearing and thus the angle of the relevant bones during use (e.g. tibia, in the case of the knee replacement), such as during a gait cycle, and also the acceleration, for instance to determine the moment at which the heel contacts the ground, enabling the placement of the estimated forces within the gait cycle.

The complete measurement system may be embedded within the instrumented orthopaedic bearing according to embodiments of the invention, and the data may be sent out wirelessly, for example using inductive coupling with two coils, of which one is located outside the body, and one inside the instrumented orthopaedic bearing or the like (FIG. 3). Power may be provided by an internal battery, and/or harvested from the movement of the knee itself, and/or through inductive transmission techniques utilising the same inductive coil pairs used for communication.

### BRIEF DESCRIPTION OF THE DRAWINGS

Further details, aspects and embodiments of the invention will be described, by way of example only, with reference to the drawings. In the drawings, like reference numbers are used to identify like or functionally similar elements. Elements in the figures are illustrated for simplicity and clarity and have not necessarily been drawn to scale.

FIG. 1 schematically shows an example of an UKR comprising an orthopaedic bearing according to embodiments of the present invention;

FIG. 2 schematically shows an example of an instrumented orthopaedic bearing according to embodiments of the present invention;

FIG. 3 schematically shows an example of the overall bearing measurement system according to embodiments of the present invention, including the electronic components embedded in the instrumented orthopaedic bearing;

FIG. 4 shows two exemplary forms of capacitive sensing elements that may be incorporated into an instrumented orthopaedic bearing according to embodiments of the present invention;

FIG. 5 shows an exemplary array layout of the capacitive sensing elements of FIG. 4 that may be utilised in an instrumented orthopaedic bearing according to embodiments of the present invention;

FIG. 6 shows an example of a TKR having a cam/post/peg arrangement according to embodiments of the present invention;

FIG. 7 shows an example coupled mathematical and Finite Element Model according to embodiments of the invention that may be used to estimate displacement and force from measured capacitance data;

FIG. 8 shows a graph of a simulated capacitance change for a capacitive sensing element array according to an example embodiment of the invention;

FIG. 9 shows the results of a real-life physical compression experiment where a femoral component exerts force on the bearing according to example embodiments of the invention;

FIG. 10 shows the compression experiment of FIG. 9, transformed using inversion, translation and scaling to estimate the displacement of the actuator;

FIG. 11 shows a lookup table used to transform estimated displacement into estimated force according to an example embodiment of the invention;

FIG. 12 shows the measured force exerted by the actuator and the estimated displacement according to an example embodiment of the invention;

FIG. 13 shows the estimated force, estimated using linear interpolation techniques and the lookup table of FIG. 11, together with the measured force of FIG. 12;

FIG. 14 shows a simplified schematic representation of a process to convert capacitance into estimated displacement and load, according to an example embodiment of the invention;

FIG. 15 shows a simplified schematic representation of a process to convert estimated displacement into estimated load, according to an example embodiment of the invention;

FIGS. 16 (a) to (e) show the complete assembly, top layer, middle layer, bottom layer and silkscreen layer respectively for use in a printed circuit implementing an array of capacitive sensing elements for use in an orthopaedic bearing according to the invention; and

FIG. 17 is a circuit diagram of the array of capacitive sensing elements shown in FIG. 16.

### DETAILED DESCRIPTION OF THE PREFERRED EMBODIMENTS

Because the illustrated embodiments of the present invention may for the most part be implemented using mechanical or electronic components and circuits known to those skilled in the art, details will not be explained in any greater extent than that considered necessary for the understanding and appreciation of the underlying concepts of the present invention and in order not to obfuscate or distract from the teachings of the present invention.

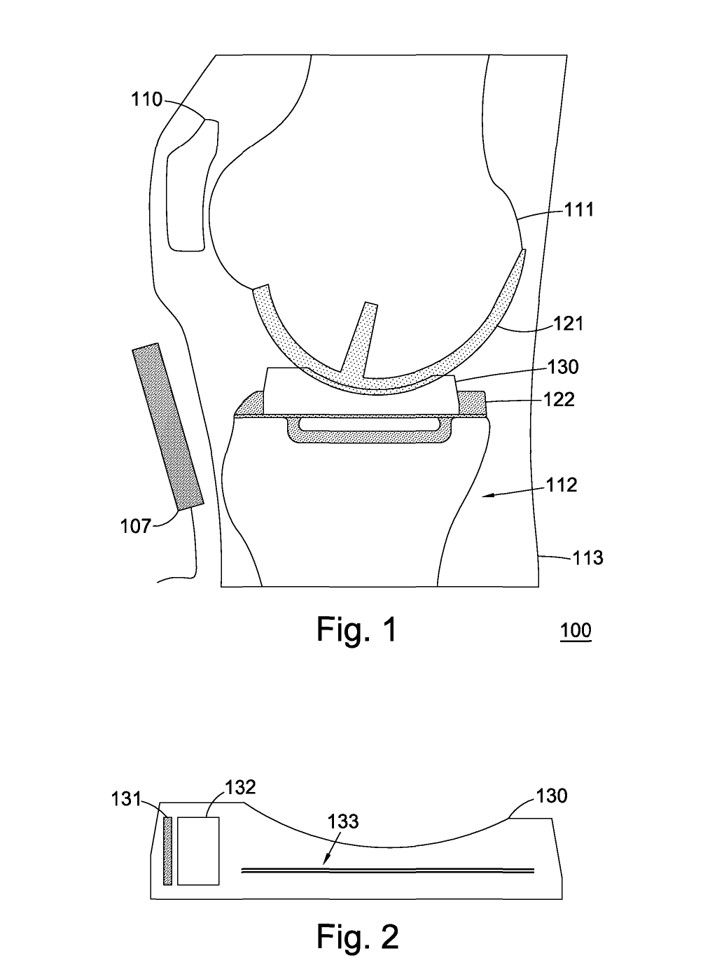

FIG. 1 schematically shows a lateral side view of an example of an instrumented UKR (100) comprising an instrumented orthopaedic bearing 130 according to embodiments of the present invention.

In FIG. 1, the femur 111 and tibia 112 of the leg having a replacement knee fitted are prepared during a surgical operation to accept a femoral component 121 and tibial tray 122, respectively. Together with the orthopaedic bearing 130 according to embodiments of the present invention, and the unaltered patella 110, they form a dynamic mechanical system within the tissue of the knee 113. An external induction coil 107, may be used to provide power and/or a communication link to the electronics within the instrumented orthopaedic bearing 130 according to embodiments of the present invention, through inductive coupling and the like. Other power and communications systems may equally be used, and the invention is not so limited.

FIG. 2 schematically shows in more detail an example of an instrumented orthopaedic bearing 130 according to embodiments of the present invention.

The instrumented orthopaedic bearing 130 comprises one or more (such as an array of) capacitive sensing elements 133 formed within the bearing 130, the capacitance(s) of which are measured by embedded electronic components 132 and may be transmitted through the skin and tissue 113 (of FIG. 1) using an internal antenna/induction coil 131. The information from the internal antenna/induction coil 131 may be received by an external antenna coil 107, which resides outside the body (as shown in FIG. 1), either temporarily or permanently.

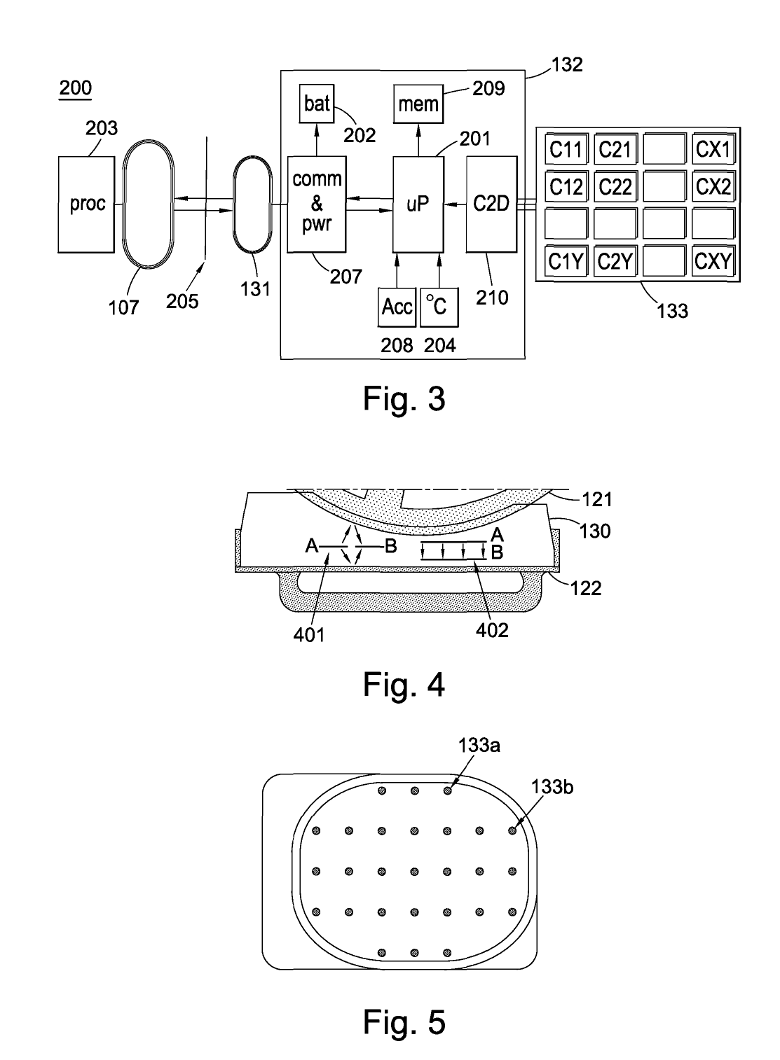

The external antenna coil 107 may also be configured to provide power to the embedded electronic components 132 (FIG. 3) using a changing electromagnetic field that may be picked up by the internal antenna/induction coil 131 located inside the instrumented orthopaedic bearing 130 (i.e. an inductive power coupling). In this way, the electromagnetic field may then be converted into power by, and for, the electronics of the bearing 132. The internal antenna/induction coil 131 is connected to the signal processing equipment located within the instrumented orthopaedic bearing 130 according to embodiments of the present invention, as exemplified by FIG. 3

FIG. 3 shows an example of the overall bearing measurement system 200 according to embodiments of the present invention, including the electronic components embedded in the instrumented orthopaedic bearing 130.

As shown, the embedded electronic components 132 may include: a Capacitance to Digital converter (C2D) 210, or any other suitable circuit, operable to read the different capacitive sensing element outputs from the capacitive sensing element array 133 and convert them into values indicative of the relative changes in capacitance of the capacitive sensing element array 133 during use. Each capacitive sensing element in the array 133 may be addressed and measured independently, with indexing between the different capacitive sensing elements in the array 133 being performed by the C2D 210, or by a multiplexer (not shown) between the C2D 210 and the array of capacitive sensing elements 133. The digital readings from the C2D may then be processed by a microprocessor (uP) 201 within the encapsulated electronics 132, or sent in raw data form to an outside processor 203 through the communications and power module 207 operably coupled to the outside microprocessor 203 through the external antenna/induction coil 107, via the inductive antenna/induction coil 131. It may be advantageous to carry out the processing outside of the instrumented orthopaedic bearing, so as to reduce cost, complexity and power usage.

Where the data is sent wirelessly, it may be transferred from the internal antenna 131 through the body boundary 205 to the external antenna 107 and on to the external processor 203. The embedded electronic components 132 may further include a temperature sensor 204, an accelerometer 208 (or any other suitable sensors), and memory 209 operably coupled to the internal processor 201. The memory 209 may be used to store any one or more of: firmware of the internal microprocessor; parameters and computer models used in the calculation to correlate capacitance changes to compression experienced by the bearing; temporarily store output readings from the capacitive sensing elements 133; and the like. The memory 209 may be any suitable memory, but most preferably non-volatile memory to reduce power usage. Exemplary forms of memory suitable for use in embodiments of the invention include, but are not limited to: Static Random Access Memory (RAM), flash RAM, phase change memory; and the like.

Power for the internal electronic components 132 may be generated by the communications and power module 207 from energy gathered from the external coil 107 via the internal antenna/induction coil 131 (i.e. by inductive power transfer). Optionally, a battery 202 may be included to power the embedded/encapsulated electronics 132 in the absence of any external power field being available, and/or with any excess energy harvested through the inductive power transfer process stored in the battery 202 for later use. Thus, typically, all the internal components are powered by the communications and power module, or, in the absence of an external inductive power transfer field, the battery 202. The battery 202 might be used to prevent loss of function due to loss of external field that supplies power, coming from the external coil 107, or in a situation where electromagnetic radiation is unavailable or undesirable, such as during a surgical procedure, where electromagnetic radiation may influence the electronics in the operating room. The system may also include a Real Time Clock (not shown) to allow the measured data to be accurately placed in time.

The encapsulated electronics 132 may be placed within a portion of the instrumented orthopaedic bearing 130 that does not undergo direct mechanical compression during use, for example, located at one end of the orthopaedic bearing 130 (as shown in the figures). Alternately, where the electronics can be formed with sufficient flexibility, they may be placed centrally within the orthopaedic bearing 130 (not shown).

An exemplary method of creating an instrumented orthopaedic bearing 130 according to embodiments of the invention is by compression moulding, for example using Ultra-High-Molecular-Weight PolyEthylene (UHMWPE) powder compacted under high pressure into a suitable shape of orthopaedic bearing 130. To ensure the embedded electronics 132 survive this manufacturing process, the bearing may be created in two stages. First, the one or more capacitive sensing elements 133, for example in the form of a foil or mesh, may be put inside the UHMWPE powder and compacted. Testing of the resultant capacitive sensing element array formation may be carried out at this point to determine a good bearing construction. The sensor foil/mesh may be located within the orthopaedic bearing 130, and one end of the foil/mesh (with the electrical contact points leading to each of the capacitive sensing elements) protruding on one side of the orthopaedic bearing 130, where a small protective cavity remains. Then, in the second stage, the electronic components 132 may be placed inside the cavity as a small module, and operably coupled (e.g. by soldering) to the foil/mesh 133. Finally, the internal antenna/induction coil 131 may be placed within the orthopaedic bearing at a suitable (e.g. distal) position and operably coupled to the electronics 132, and the whole cavity is sealed off hermetically, possibly with UHMWPE or a different material.

### Sensor Arrangement

A capacitor, consisting of two parallel, identically sized conductive plates, can be described by the following equation (Eq 1):

$$C = \frac{ε 0 * ε r * A}{d}$$

where:

### C Capacitance [in Farads=F];

∈0 Dielectric permittivity of a vacuum [in F/m], e.g., 8.85E−12
∈r Relative dielectric permittivity for a particular material used between the plates [dimensionless]
A Area of plates that is parallel to each other [in m²];
d Distance between plates [in m].

According to Eq 1, when the distance between the plates of a capacitor decreases, then the measured capacitance increases. Therefore, when the plates of the capacitive sensing element are aligned so that the distance between the plates changes under mechanical load of the orthopaedic implant on the instrumented orthopaedic bearing 130, measured capacitance changes are in accordance with load variation on the bearing 130.

The capacitive sensing elements 133 used may comprise a number of different configurations. FIG. 4 shows (in side view) two of the possible configurations: a) two adjacent capacitor plates 401 in the same plane (horizontal, when viewed from the side, as per FIG. 4) that form a capacitive sensing element in combination with the metallic components of the overall orthopaedic implant above 121 and below 122 the plates; b) two parallel capacitor plates 402 adjacent one another, but separated by a distance perpendicular to the plates that form a typical parallel plate capacitor. In both cases, the straight arrows around the plates (A and B) show the electrical path of the electric field between the plates. In the case of adjacent plates 401, the shortest path between plates A and B, via the metallic component 121 or 122, will provide the value for d, in Eq 1 above. In the case of parallel plate 402, the distance between the parallel plates will form the shortest path determining d. Each of the capacitors in the capacitive sensing element array 133 may be one of the two examples, or another suitable type of capacitive sensing element configuration, i.e. in the case of an array being used, it may be homogenous or heterogeneous.

In the case adjacent plates 401, it is important to shield the leads (not shown) connected to the plates, so as to prevent premature coupling of the common electrode and sensing electrodes, limiting coupling solely to the parts not covered by shielding.

In the parallel plate configuration of the capacitive sensing element 402, the material between the plates may be UHMWPE, but may also be another polymer that is easier to produce into a thin layer, and/or having more suitable or better known compression characteristics over the expected temperature range to be experienced by the instrumented orthopaedic bearing 130 during use (in vivo).

In the case of a small change in capacitance, capacitance change is linearly related to deformation, or the change in d, and when capacitance is expressed as a percentage change, all constants (∈0, ∈r and A) can be removed from the formula, and a compression of Δd results in the following capacitance change, according to Eq 2:

$$C ( i , j ) = ( \frac{1}{d - Δ d ( i , j )} - \frac{1}{d} ) \times d \times 100 [ \% ]$$

where:
C(i,j) is the change in capacitance for every sensor coordinate i, j [in %]
d is the distance between the two plates of the sensor [in m];
Δd(i,j) is the distance change between the two plates [in m].

FIG. 5 shows an exemplary array layout of the embedded capacitive sensing elements of FIG. 4 that may be utilised in an instrumented orthopaedic bearing 130 according to embodiments of the present invention. Spatially, the capacitive sensing elements may be arranged in a grid array as depicted in FIG. 5, but may also be arranged in a hexagonal or round pattern (e.g. concentric circles). Each of the capacitive sensing elements (133a, 133b) may be arranged as either the adjacent or parallel plate variations (as shown in FIG. 4), or may be alternative capacitive arrangements instead.

The capacitive sensing elements need not each have two separately addressable electrodes. Instead, multiple capacitive sensing elements may be made with one common electrode and one uniquely addressable electrode, or be formed in a (perpendicular) horizontal row and vertical column layout (the directions being when viewed from above, straight on), in which the point where the metal of each row and column overlap forms the respective capacitive sensing element, and is addressed using the respective row and column lines.

By using an array of capacitive sensing elements, it is possible to calculate the Centre Of Pressure (COP), i.e. the point where the maximum force is put on the bearing, and also the pressure distribution within the orthopaedic implant as a whole, and in particular the orthopaedic bearing 130, both of which allow improved assessment of the functionality of the fitted orthopaedic bearing 130, the patient gait, and the like.

Although the above-described examples illustrate the use of an instrumented orthopaedic bearing 130 according to embodiments of the invention in a UKR, embodiments may also be used in other types orthopaedic implants, such as, for example, a Total Knee Replacement (TKR), a hip replacement, or any other joint replacement without any substantial differences other than the shape of the bearing (e.g. in a hip joint replacement, the bearing may be substantially hemispherical, rather than flat). In either case, the output from an array of capacitive sensing elements will provide an accurate indication of COP and pressure distribution regardless of the form of the bearing 130 itself.

Determining COP is particularly important when estimating the extent and location of wear, especially delamination: a fault condition when layers of the polymer (e.g. UHMWPE) detach from the bearing and leave a cavity, which can deteriorate over time leading to premature failure of the orthopaedic implant. Delamination can be detected by an array of capacitive sensing elements according to embodiments of the present invention in two ways: (1) When using two adjacent electrodes 401 with a cavity directly above the sensor, there is less material with a fixed relative dielectric constant, characteristic for UHMWPE. Therefore, if there is conductive body fluid in the cavity, the electric path between the two electrodes (301) will be shorter, and the measured capacitance will be increased locally, compared to the surrounding electrodes. (2) When using a parallel sensor 402 with a cavity directly above the sensor, the material between the sensor plates will compress less, compared to the sensors above which there is material above the sensor and touching the metal components surrounding the bearing. Therefore, there will be localized areas of decreased compression that can only be explained by holes in the bearing.

Delamination and associated thinning of the bearing can mean there is an increased risk of exposure of the sensors to the bodily fluids. There are several ways to reduce that risk:

(1) Make the capacitive sensing element(s) very thin, so that there is relatively more UHMWPE that needs to be worn before the sensors are exposed.
(2) Place the capacitive sensing element(s) exactly in the middle of the bearing (side view), assuming that wear rate is identical on all sides of the bearing.
(3) Place the capacitive sensing element(s) spatially/radially away from the centre of the bearing (top view), where most of the force is centred. A possible way to position the sensors is in a circle surrounding an estimated COP, or even as a set of concentric circles.

As mentioned above, the instrumented orthopaedic bearing 130 may include other sensors apart from the capacitive (compression/tension detection) sensors. For example, a temperature sensor 204 may be included, to measure temperature at the time of the other sensor readings being taken. This may be particularly useful when using orthopaedic bearings formed from materials having temperature dependent elastic moduli. For example, with Ultra High Molecular Weight Poly Ethylene (UHMWPE), the elastic modulus of UHMWPE may decrease by up to 46% when the temperature changes from 20 degrees Celsius to 37 degrees Celsius (i.e. in the temperature range experienced when the bearing is implanted in a human body), increasing the capacitance change under identical load. Therefore, in order to get accurate measurements, the temperature is advantageously measured and incorporated into the deformation calculation model.

Another useful additional sensor is an accelerometer 208, which may be used to accurately position the measured capacitance temporally within the gait cycle. For instance, using an accelerometer 208, the moment the heel touches the ground produces a characteristic deceleration pulse, therefore can be used to determine the point of contact. Furthermore, the gravity component may also be measured, allowing the measurement of the angle of the accelerometer 208 to the ground, from which the rotation of the orthopaedic implant (or relevant bone structures) to the ground may be determined. For example, since the instrumented orthopaedic bearing 130 is typically parallel to the tibial component 122, the angle of the tibia can be determined. The combined information regarding acceleration and position allow for a full temporal positioning of simultaneous capacitive measurement within the gait cycle, therefore allowing an improved temporally-dependent analysis of the wear of the orthopaedic bearing and the like.

Further optional components 209 might include a mass storage device to store firmware for the microprocessor 201, or data coming from the C2D 210. The mass storage may be used in a situation where communication may be unavailable or undesired, but where either there is sufficient power in the battery 202, or coming in through an external field, supplied by the external coil 107.

### Modelling

The capacitive data, position of the bearing, position of the capacitive (or other) sensors, localized temperature(s), accelerometer readings, and so on are typically inputted into a combined Mathematical and Finite Element model that calculates deformation using Eq 2, from which is it possible to calculate the resulting COP and the amount of force necessary to get the calculated deformation of the instrumented orthopaedic bearing 130. The model may also be used initially to determine the best position for the electrodes/capacitive sensing elements within the instrumented orthopaedic bearing 130, and may be altered over time to take into account new parameters of the orthopaedic implant or parameters to be assessed, etc. Typically, the model may be run on a computer, during post-processing, after the measurements are finished.

Embodiments of the present invention provide advantages over the prior art bearings. These include more accurate measurement of pressure distribution in the replacement orthopaedic joint, compared to other types of orthopaedic measurement devices, which have used strain gauges to measure deformation of the metallic components of a knee replacement. However, although the COP can be calculated by using multiple strain gauges, they cannot be used to determine the accurate pressure distributions that an array of capacitive sensing elements 133, according to embodiments of the present invention, may provide. Furthermore, embodiments of the present invention are capable of accurately determining where each respective component of the overall joint is located relative to the others. For example, in a replacement knee joint, it is possible to determine the distance of the femoral component 121 relative to the tibial component 122, which aids in assessing the quality of operation of the replacement knee, and/or the operation that installed it, as well as measurement of kinematic data.

Another advantage over previous bearings described in the art, include a reduced size of bearing. The use of strain gauges requires a large, bulky and expensive implant. For example, strain gauge instrumented knee implants formerly have been implemented in the tibial component of the knee, extending into the core of the tibia itself, whereas the current capacitive sensing element measurement system can be integrated within the small confines of the orthopaedic bearing alone. The instrumented orthopaedic bearing of the present invention may be left in place, even after the assessment period is complete, because the instrumented orthopaedic bearing 130 according to embodiments of the invention does not operate differently to a normal bearing, and can be largely inert. This means embodiments of the present invention may be used on every patient, rather than just a test group. Moreover, since the instrumented orthopaedic bearing may be used in all cases, continuous data collection and assessment is possible (over the life of a single bearing, or over multiple bearings in a patient group) which can provide improved data sets for the further development of orthopaedic implants as a whole, for example improving designed of the respective articulating joint, bone inserts, and the like. It would also allow continuous assessment of the replacement orthopaedic implant over time, thereby providing tailored care and implant assessment to the patient, and may provide further functionality such as step counters, and the like.

Another advantage of the current invention is the lack of substantial change in mechanical behaviour of the replacement joint compared to original joint. If care is taken that the capacitive (or other) sensors are not too thick, then they pose no noticeable mechanical resistance to the orthopaedic bearing 130 during use, so the mechanical behaviour of the orthopaedic implant as a whole does not noticeably change. That means that existing orthopaedic components (e.g. the fixed components—femoral component or tibial component in the case of a knee replacement) will require a less complex testing and verification process, which results in a big reduction of time and money spent on the replacement joint and the subsequent assessment of the replacement joint operation. Furthermore, because the capacitive sensing elements are sufficiently small and deformable (e.g. a foil that is implanted within the bearing material itself), it also means that the measured deformation of an instrumented orthopaedic bearing 130 according to embodiments of the present invention is equivalent to the deformation that would occur in a bearing without any sensors. In summary, the capacitive sensing elements measure exactly how much the measured material deforms, without noticeably influencing the instrumented orthopaedic bearing's mechanical behaviour.

A further advantage of using capacitive sensing elements within the instrumented orthopaedic bearing 130 is that the actual deformation of the orthopaedic bearing 130 is measured. This is because, typically, there are only small changes in deformation of the orthopaedic bearing 130 according to the embodiments of the present invention, and thus only small changes in the capacitance change. When the changes in capacitance are small, the relationship between deformation and capacitance change is linear, therefore, changes in capacitance correlate in a linear fashion to changes in compression. Therefore, the measured capacitance changes can be interpreted directly as mechanical compression and used as an input into the mechanical model of the orthopaedic bearing 130.

No galvanic contact will occur between the metals or electronics of the instrumented orthopaedic bearing 130 and the body. Unlike known orthopaedic devices, all the components of the instrumented orthopaedic bearing 130, according to embodiments of the present invention, are embedded/encapsulated within the bearing polymer material (e.g. UHMWPE), or another suitable non-conducting material. Therefore, there is no electrical contact between any of the metals of the measurement circuit, and there are no problems that arise from insertion of metallic components within the body, such as corrosion and rejection.

Consummate with the reduced complexity of the instrumented orthopaedic bearing of the present invention, the disclosed capacitive measurement system can be built with commercially available components, resulting in a total cost of an instrumented orthopaedic bearing 130 according to embodiments of the present invention being several orders of magnitude less costly than custom instrumented orthopaedic implant devices.

Applications for the instrumented orthopaedic bearing 130 may include:
1. Gait Study—embodiments of the present invention allow the accurate measurement of forces that act on orthopaedic bearings/implants as a whole during use. Therefore, recipients of the instrumented orthopaedic bearing 130 according to embodiments of the present invention can be offered tailored recovery and physiotherapy to ensure that their gait is improved. For instance, they could be taught how to walk with reduced heel strike.
2. Outlier detection—when the data of multiple instrumented orthopaedic bearings 130 is grouped, a set of standard/typical parameters for patients (e.g. regarding maximum force and deformation) can be formed, thereby producing a set of standard usage parameters that enable the detection of outlier (i.e. abnormal) results in patients, providing early indication of problems with the joint replacement. Outlier detection can then be used to detect an orthopaedic bearing that is operating differently from the others early on, so that patient can then be examined more thoroughly to determine if there is a significant problem. This will result in fewer unexpected orthopaedic bearing failures and fewer, less drastic revision operations.
3. Verifying correct implantation procedures—during an implantation operation, the instrumented orthopaedic bearing 130, according to embodiments of the present invention, may provide evidence as to the correctness of the implant procedure. When there is proof that a bearing/implant/bone insert has or has not been properly implanted, rectifying action can be taken immediately, and/or insurance claims can be thwarted or reduced. Furthermore, if a surgeon gets real-time feedback from the instrumented orthopaedic bearing within the implant during an operation regarding, for instance, static force; the fixation or position of the bone insert portions of the implants may still be adjusted during the same surgical procedure, rather than requiring an extra operation later.
4. Simple on-going integrity check of the implant—when an implant operation has been performed correctly, an implant should last at least 10-15 years. Traditionally, a patient would go to the hospital for a check-up using an x-ray imaging device to check alignment and wear. This has all the attendant problems of exposure to radiation, taking up x-ray resources (machine and operator), and the like. In contrast, when using an instrumented orthopaedic bearing 130 according to embodiments of the present invention, wear can be checked in an on-going fashion more accurately and easily by the capacitive sensing elements inside the orthopaedic bearing 130, and without having to expose the patient to potentially hazardous x-rays. Moreover, because of the increased possibility of detecting localized delamination and wear in situ/in vivo, orthopaedic implant degradation can be detected at an earlier stage, which enables preventive rather than corrective procedures to be applied.

Other applications may include: the ability to run diagnostic assessments of the replacement joint outside of the hospital environment (and even continuously during use), such as in the patient's home; the provision of usage statistics, such as average force estimations, usage outside of recommended parameters, walking step counters, etc.

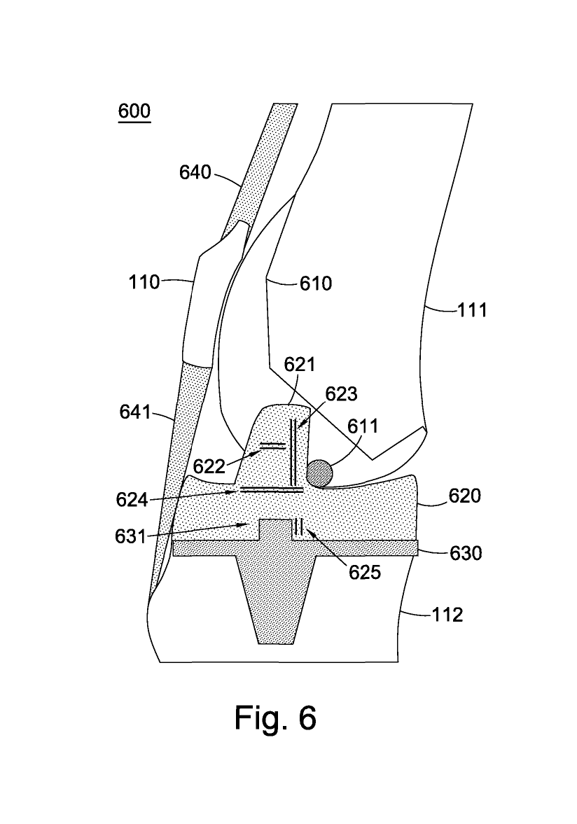

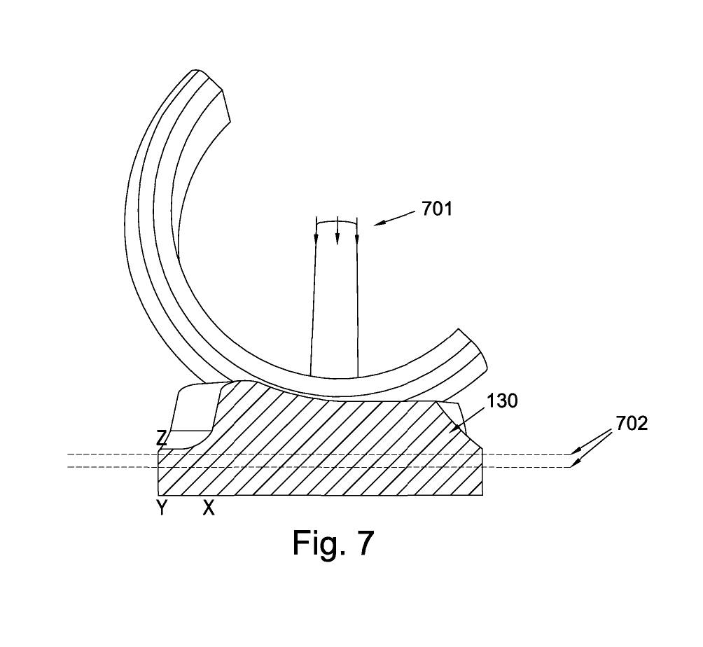

Additionally or alternatively, but not limited to a total knee replacement, the capacitive sensing elements may be located in areas where high stresses are expected. To illustrate this, measurement of increased stress regions in case of a Total Knee Replacement with a cam/post/peg will be explained, with reference to FIG. 6.

In a healthy knee, and in the Uni-compartmental Knee Replacement, both the anterior and posterior cruciate ligaments are functional and intact. Because cruciate deficiency leads to abnormal kinematics, affecting activities of daily life and reducing functional capacity of the knee joint, many total knee replacements have an articulating system with a cam 611 and a post 621, designed to keep the femur from translating anteriorly on the tibia.

The TKR may comprise fixed parts. For example, at the distal end of the femur 111, there may be a femoral component 610 with a cam 611, so that when the femur 111 moves in the anterior direction, the cam 610 touches the post 621, which prevents further movement in the anterior direction. The post 621 may be a component of the bearing 620, which may be attached to the tibial tray 630, which in turn may be firmly fixed to the tibia 112. In this example, the femur may be prevented from moving up because of tension provided by the quadriceps tendon 640 and patellar tendon 641, which are connected to the quadriceps muscle (not shown), the patella 110 and the tibia 112.

There are several places where there may be increased stress in the bearing. Firstly, there may be compression of the bearing 620 between the femoral component 610 and the tibial tray 630 (similar to that described above in relation to UKR). Secondly, there may be deformation of the post 621 because the forward motion of the femur 111 results in a transfer of force through the cam 611. Thirdly, the forward motion of the femur may also result in forces acting upon the peg 631, the peg 631 being a part of the tibial tray 630 at the bottom that grips in a slot of the bearing 620.

In alternative examples of the present invention, these additional deformations can be measured by embedding capacitive sensing elements in appropriate portions of the bearing 620, in either a parallel or adjacent configuration, as described in FIG. 4. Several example positions (items 622-625) are shown in FIG. 6, in horizontal or vertical orientations. The example of measuring compressive deformation through use of capacitive sensing elements parallel to the bottom of the bearing, as described above is still applicable but not shown, because the situation is similar to that described previously for the uni-compartmental bearing.

Example horizontally oriented capacitive sensing elements 622 may comprise at least two parallel plates that move sideways relative to one another due to shearing of the post 621. In case of shear, anterior translation is higher at the top of the post, than at of the base of the post. Therefore, capacitive plates 622 positioned parallel to the base of the post may each have a different height above the base of the post and thus a different anterior translation. So, in case of shear occurring, the overlapping electrode area A (Eq 1), rather than the distance between the plates d, reduces, with more shear resulting in a reduced area and hence reduced capacitance.

Example vertically oriented capacitive sensing elements 623 may measure the deformation of the post 621 resulting from the force of the cam 611 acting on it. Example horizontally orientated capacitive sensing element grid 624 may measure deformation of the base of the post 621. In case of shear of the post 621 because of pressure provided by the cam 611, the base of the post 621 located most distally away from the cam 611 undergoes compression, while the base of the post 621 located proximally to the cam 611 undergoes less compression or increased tension. Therefore, when comparing multiple capacitive readings from the capacitive sensing element grid 624 (and/or sensors 622 and 623) a measure of the deformation of the post 621 can be obtained. Example vertically orientated capacitive sensing element(s) 625 may measure deformation of the bearing caused by the peg 631. Although example capacitive sensing element(s) 625 are shown on the right hand side of the peg 631, they may also be located anywhere else next to, or around, the peg 631.

In the situations described above, the capacitive sensing elements are not necessarily aligned in a plane parallel to the bottom of the bearing, but may be distributed in the perpendicular (vertical) axis in respect to the bottom of the bearing. A two or three dimensional grid using either vertically or horizontally, or a combination of horizontally/vertically, orientated capacitive sensing elements may also be used.

### Measurements

Since all the components may be encapsulated within the instrumented orthopaedic bearing 130 itself, embodiments of the invention provide the capability to more readily replace the orthopaedic bearing if incorrect functioning of the orthopaedic implant as a whole is detected, or failure of the bearing is detected, such as through wear, delamination of the polymers, and the like. In the case of detection of a situation in which premature failure is likely, the corrective surgery is likely to be less severe, compared to previously known orthopaedic implants that incorporate the electronics into the bone portions themselves.

FIG. 7 shows an example coupled mathematical and Finite Element Model according to embodiments of the invention that may be used to estimate displacement and force from measured capacitance data. In this figure, the compressive force is represented by arrows 701 on the femoral component, and the dashed line planes 702 represent the electrodes of the capacitance. The capacitance electrode area may cover the entire transverse plane of the bearing.

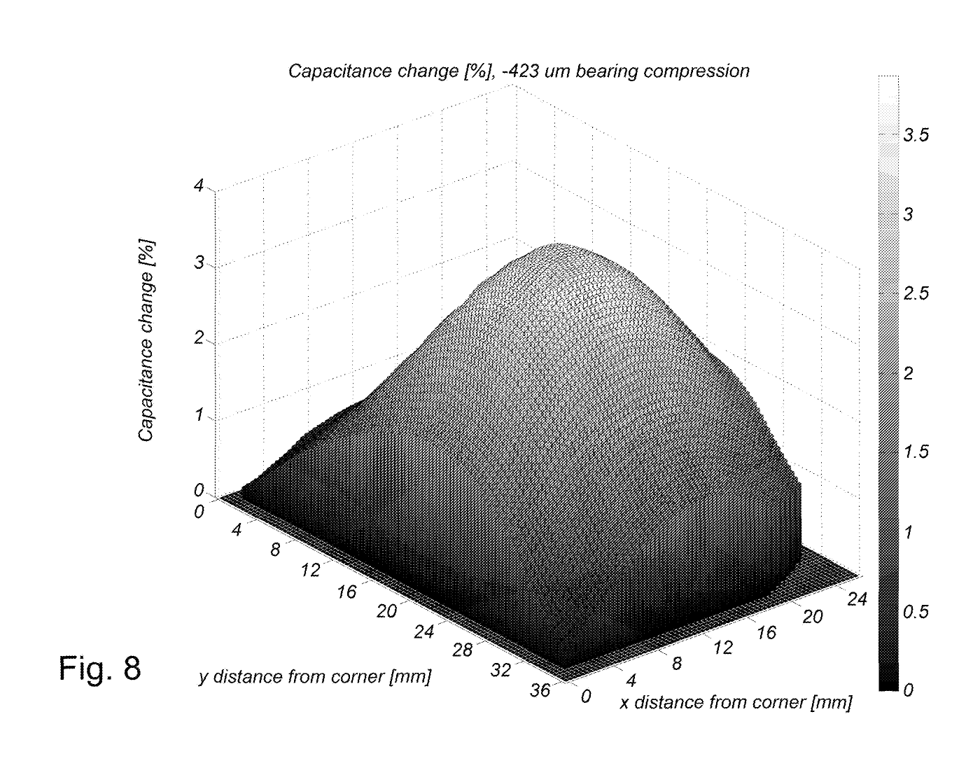

FIG. 8 shows a graph of a simulated capacitance change for a capacitive sensing element array according to an example embodiment of the invention. In particular, this figure shows a simulated capacitance change for a simulated capacitance array of 100 by 71 capacitive sensing elements 133. Any shape of capacitor can be simulated by integrating several indexed sub-capacitors. The capacitance change of a capacitor that spans the entire transverse plane can be simulated by integrating all the capacitive changes of the sub-capacitors.

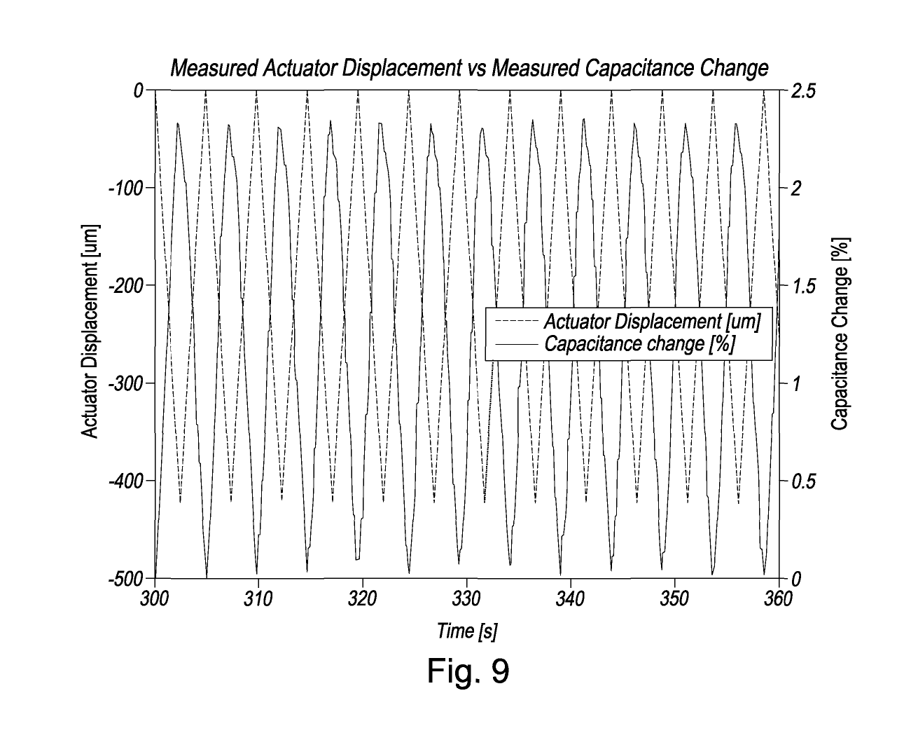

FIG. 9 shows the results of a real-life physical compression experiment where a femoral component exerts force on the bearing according to example embodiments of the invention. The maximum measured displacement of the femoral component in this experiment is 423 μm, and the maximum capacitance change is 2.3%. The capacitor in this experiment covered the entire transverse plane of the bearing, identical to the simulated model bearing, shown in FIG. 7. For this instance, the dielectric material of the capacitor was formed from the same UHMWPE as the bearing itself.

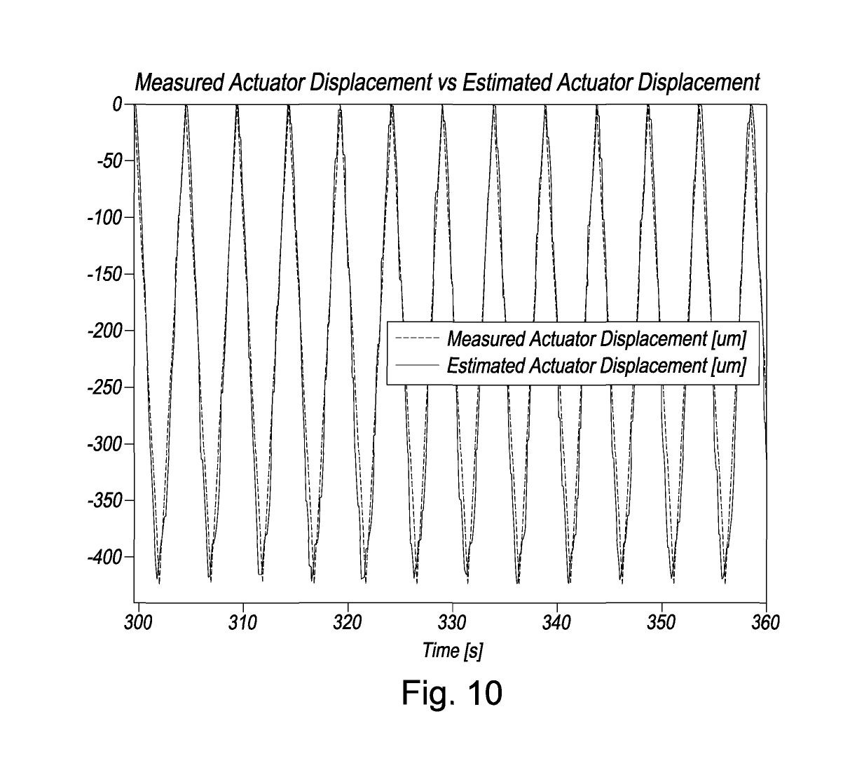

FIG. 10 shows the same compression experiment as shown in FIG. 9, but where the change in capacitance is transformed using inversion, translation and scaling to estimate the displacement of the actuator.

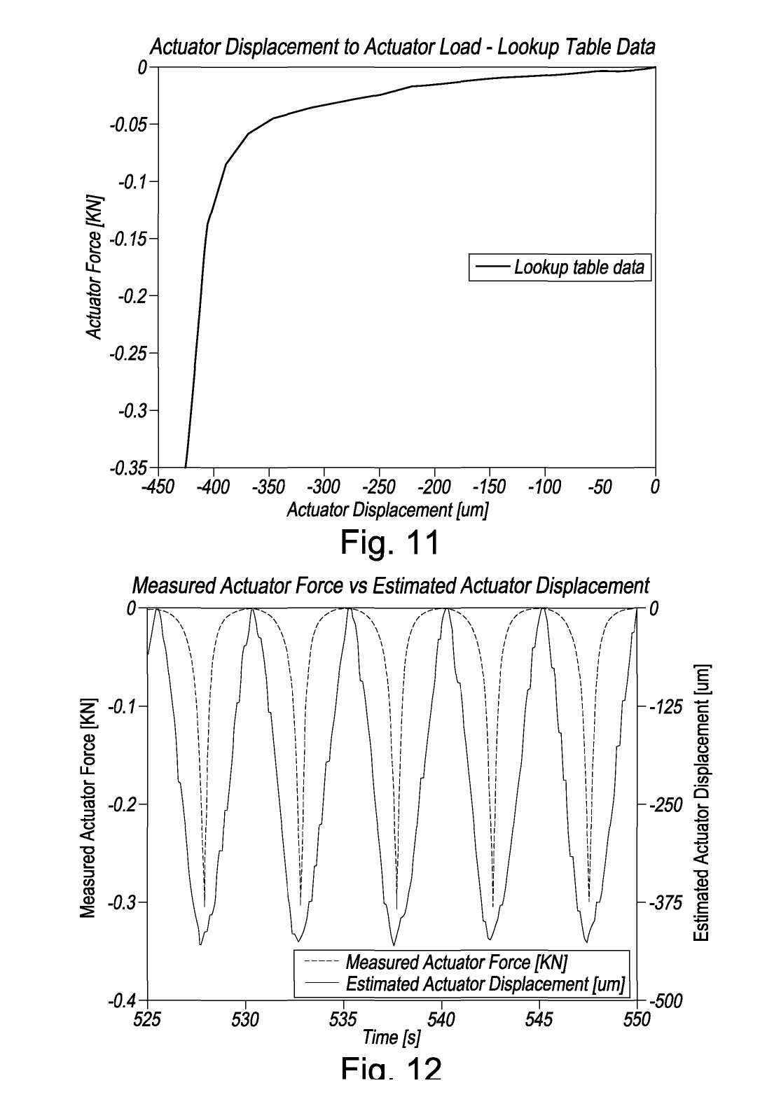

FIG. 11 shows the lookup table used to transform estimated displacement into estimated force according to an example embodiment of the invention.

FIG. 12 shows the measured force exerted by the actuator and the estimated displacement according to an example embodiment of the invention.

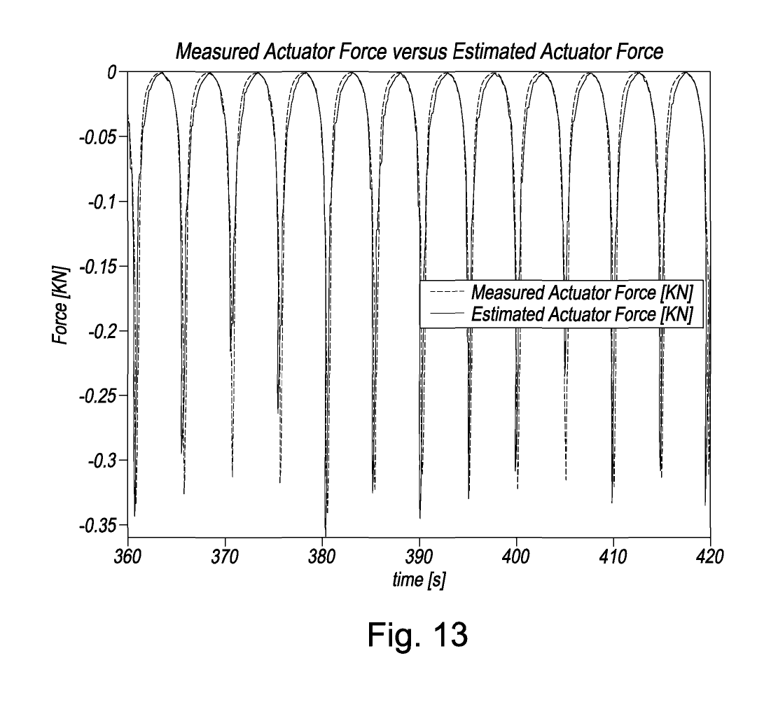

FIG. 13 shows the estimated force, estimated using linear interpolation techniques and the lookup table of FIG. 11, together with the measured force of FIG. 12.

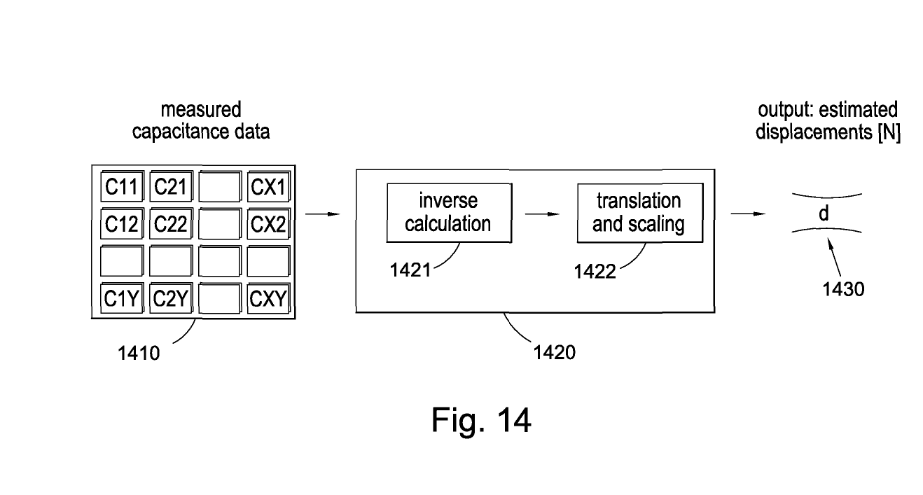

FIG. 14 shows a simplified schematic representation of a process to convert capacitance into estimated displacement and load, part of the “coupled mathematical and Finite Element Model”, according to an example embodiment of the invention.

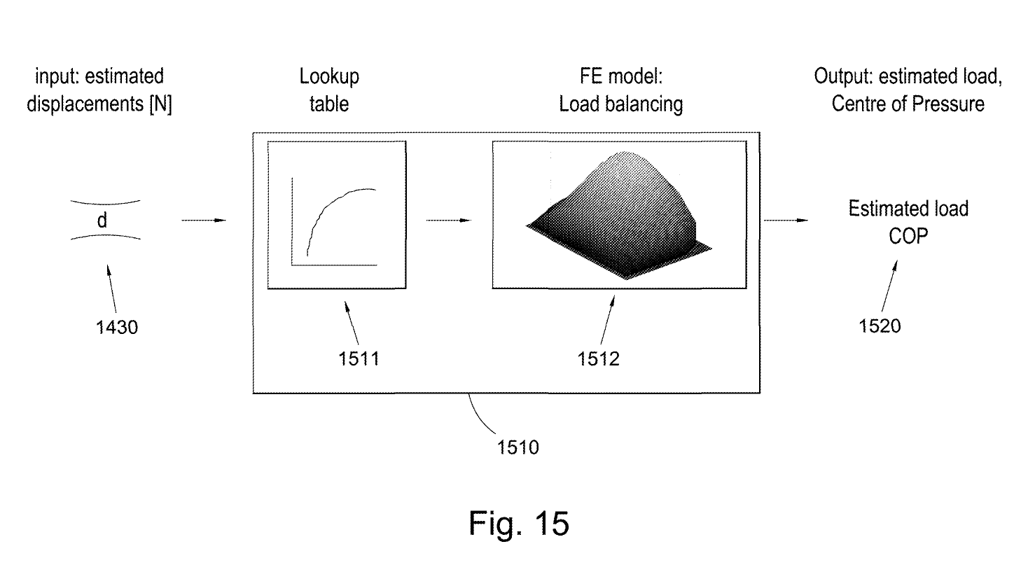

FIG. 15 shows a simplified schematic representation of a process to convert estimated displacement into estimated load, part of the “coupled mathematical and Finite Element Model”, according to an example embodiment of the invention.

The process to estimate force and displacement from measured capacitance is described in FIG. 14 and FIG. 15, and functions as follows:

Measured capacitance data 1410, (see FIG. 9) may be transformed into estimated displacement 1430, by solving Eq 1 (using an inverse calculation 1421) for d using the measured capacitance, constants ∈0 and ∈r, and A, which in this example is approximated by a constant. The result of that calculation may be transformed and scaled 1422 using calibration data (not shown), which may be gathered during calibration of the instrumented orthopaedic bearing, as described previously. The process of converting measured capacitance into estimated displacement 1430 is shown in FIG. 14. Estimated displacement 1430 is shown in FIG. 10.

The estimated displacement 1430 may be further transformed into estimated force 1520 by using linear interpolation combined with a lookup table 1511 (FIG. 11).

The resulting estimated displacement may be input into the Finite Element model 1512, in which the displacement is compared with the displacement from other sensors of the capacitive sensing element array, and in which the load is balanced across the bearing model. The result of the transformation from displacement to force is shown in FIG. 12, and FIG. 13. Note that for this example, the lookup table 1511 data is gathered using data between 360 and 420 seconds of the experiment shown in FIG. 9 and the force is estimated using data between 300 and 360 from the same experiment FIG. 13, illustrating that the model works well if the model parameters are constant. If desired, it would also be possible to use a simpler method of force estimation by creating a lookup table to estimate force directly from capacitance change. This is particularly beneficial in the case of using one sole capacitor, but may be less accurate.

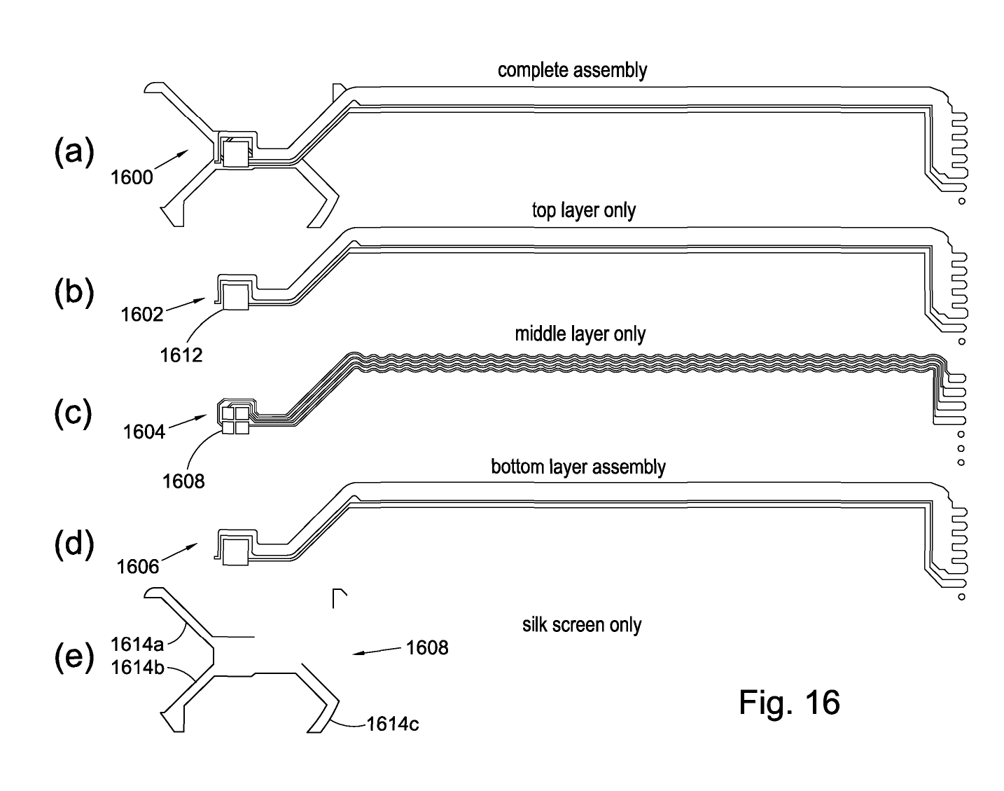

Referring now to FIGS. 16(a) to (e), there is shown the artwork for preparing a printed circuit to implement an array of capacitive sensing elements for use in an orthopaedic bearing according to the invention. FIG. 16(a) shows a view of the complete assembly 1600 that will be implemented as a flexible printed circuit. The assembly 1600 comprises three layers of conductors, a top layer 1602, a middle layer 1604 and a bottom layer 1606, alternately layered with a flexible medical grade insulating substrate, such as Polyimide The middle layer 1604 comprises four spaced apart square metal plates 1608, arranged to form a larger square, each having a single connector lead 1610 connected thereto. The connector leads 1610 link the array of capacitive sensing elements to outputs for sensing and control. The connector leads 1610 follow a partially undulating path.

The top layer 1602 comprises a fifth capacitive sensing element 1612 in the form of a larger plate, which in combination with the four spaced apart square metal plates 1608, forms four parallel plate capacitors with the fifth element being common to each capacitor. The fifth capacitive sensing element 1612 is connected to the output from the array of capacitive sensing elements to the capacitive-to-digital converter (not shown), also known as the exciting signal. Using five connector leads in this way, it is possible to measure four capacitive sensors independently, in a sequential manner.

The bottom layer 1606 comprises shielding, forming a backplane, so as to shield the connector leads to the capacitive sensing elements from external electromagnetic fields. The connector leads 1610 are said to be connected to the ‘cold-side’ of the capacitors. In this way, the connector leads 1610 are in effect protected by a shielding layer on both sides thereof. The shielding layer is grounded, or is at the exact same level at the output signal. In this way, since the shielding is covering both sides of the sensitive return lead, the forms an effective faraday cage, improving the signal to noise ratio of the capacitive sensors. The shielding also shields the electrodes from nearby metallic components, e.g. the femoral component and tibial plate. If not properly shielded, and depending on the measurement method, an additional electric path may form between the common electrode lead, the metallic objects, and the sensing electrodes, which may change the capacitive signal. In case of implementing the capacitive sensor using the method shown in FIG. 4, adjacent electrodes (401), the shield is an important part of the physical layout; preventing premature coupling of the common electrode and sensing electrodes, limiting coupling solely to the parts not covered by shielding.

In use, the array of capacitive sensing elements is embedded in an orthopaedic bearing (not shown) according to the invention. Typically, such an orthopaedic bearing is manufactured by compression moulding Ultra High Molecular Weight PolyEthylene (UHMWPE) in a negative mould. Initially, a portion of UHMWPE power is inserted into the mould, then the flexible printed circuit comprising the array of capacitive sensing elements is inserted into the mould and the remaining UHMWPE power is inserted into the mould. Then a plunger is used to apply heat and pressure so as to convert the powder into a solid bearing, having an array of capacitive sensing elements embedded therein. The connector leads are long so as to allow control over positioning of the array in the bearing. The undulating path taken by the connector leads 1610 allows them to stretch slightly without breaking during the moulding process.

The substrate surrounding the array is shaped to further assist in correctly positioning the array within the bearing. The final shape is shown in FIG. 16 (e), where the shaped to be cut is printed onto the substrate. The shaped substrate forms three locating legs 1614, projecting from the corners of the substantially square central module formed by the array of capacitive sensing elements. The legs project at approximately 45° from each side of the square module adjacent thereto. The shaped substrate provides three locating legs, while the path of the connector leads provides a fourth leg at the top right of the module. The top left locating leg 1614a has a slightly bulbous distal end, with a curved edge, designed to fit a large radius corner of a UKR bearing. The bottom left locating leg 1614b has a larger bulbous distal end, with straight edges. The bottom right locating leg 1614c is angled midway in at approximately 45°, allowing the leg to cover as much of the curved corner of the bearing as possible, while attempting to minimise leg surface area. In this way, the corner is covered and the rotation of the entire array is reduced. The locating legs illustrated allow the array to be centred within an orthopaedic bearing for use in a UKR. The person skilled in the art will understand that leg shape may be adapted to fit other bearings as required.

It will be understood that the version of connector leads shown in FIG. 16 is for use in testing, and that clinical devices will have shorter leads. Preferably, the leads will terminate in the corner of the orthopaedic bearing. The connecting leads will still comprise at least an undulating portion.

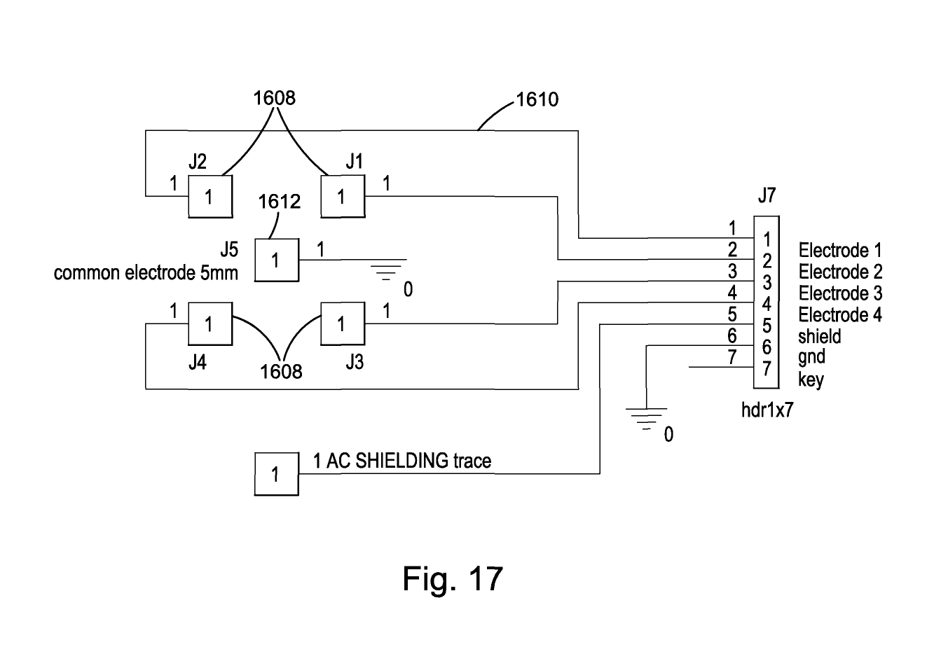

Referring now to FIG. 17, there is shown a circuit diagram of the array of capacitive sensors of FIG. 16, where similar parts have been given the same reference numerals as previously. There are shown the four sensing electrodes 1608, J1, J2, J3 J4, and the common electrode 1612, J5. J5 is connected to ground, connector J7 also has a connection to ground, such that the common electrode J5 is connected to pin 6 of J7. J7 connects to the Capacitive to Digital converter (C2D) (not shown).

In the foregoing specification, the invention has been described with reference to specific examples of embodiments of the invention. It will, however, be evident that various modifications and changes may be made therein without departing from the broader scope of the invention as set forth in the appended claims.

The connections as discussed herein may be any type of connection suitable to transfer signals from or to the respective nodes, units or devices, for example via intermediate devices. Accordingly, unless implied or stated otherwise, the connections may for example be direct connections or indirect connections. The connections may be illustrated or described in reference to being a single connection, a plurality of connections, unidirectional connections, or bidirectional connections. However, different embodiments may vary the implementation of the connections. For example, separate unidirectional connections may be used rather than bidirectional connections and vice versa. Also, plurality of connections may be replaced with single connections that transfers multiple signals serially or in a time multiplexed manner. Likewise, single connections carrying multiple signals may be separated out into various different connections carrying subsets of these signals. Therefore, many options exist for transferring signals.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures can be implemented which achieve the same functionality.

Any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

However, other modifications, variations and alternatives are also possible. The specifications and drawings are, accordingly, to be regarded in an illustrative rather than in a restrictive sense.

The terms “horizontal”, “vertical” and other orientation based terms are used merely to indicate relative positions, as viewed from a particular, natural view point, such as from above or below. This is to say, these terms are not necessarily related to the respective positions or orientations of the respective portions during use or otherwise. These terms are not to be construed restrictively.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

Unless otherwise stated as incompatible, or the physics or otherwise of the embodiments prevent such a combination, the features of the following claims may be integrated together in any suitable and beneficial arrangement. This is to say that the combination of features is not limited by the claim forms, particularly the form of the dependent claims.

1. An orthopaedic bearing suitable for use with an orthopaedic implant, the orthopaedic bearing comprising at least one deformable portion, the at least one deformable portion being formed from a flexible bearing material adapted to deform under load, characterised in that at least one capacitive sensing element is embedded within the deformable portion, the capacitive sensing element being operable to measure a change in capacitance resultant from a deformation of the deformable portion.

2. The orthopaedic bearing of claim 1, comprising a plurality of capacitive sensing elements arranged within the at least one deformable portion of the orthopaedic bearing and operable to measure changes in capacitance over an area of the bearing resultant from a deformation of the deformable portion of the orthopaedic bearing during use.

3. The orthopaedic bearing of claim 2, wherein the plurality of capacitive sensing elements comprises an array, and wherein the array is formed as a grid in a regular or irregular pattern.

4. The orthopaedic bearing of claim 2 or 3, wherein the plurality of capacitive sensing elements are distributed in three dimensions, length, width and height, of the orthopaedic bearing.

5. The orthopaedic bearing of any of claim 3 or 4, wherein the array is a square or circular pattern, and a centre of the square or circular pattern is at an estimated Centre Of Pressure.

6. The orthopaedic bearing of any preceding claim, further comprising an accelerometer operable to provide acceleration data for the orthopaedic bearing during use, in order to correlate position of knee with capacitance changes in the at least one capacitive sensing element and/or correlating capacitance changes with acceleration of the knee.

7. The orthopaedic bearing of any preceding claim, further comprising a temperature sensor operable to measure a temperature of the orthopaedic bearing during use, said temperature measurement being used to compensate for variation in the measured capacitance due to temperature change.

8. The orthopaedic bearing of any of claims 2 to 7 inclusive, further comprising a plurality of temperature sensors, operable to determine point temperatures of the bearing material across an area of the orthopaedic bearing, wherein the points correspond to capacitive sensing element locations.

9. The orthopaedic bearing of any preceding claim, further comprising electronic circuitry encapsulated within a portion of the orthopaedic bearing that does not experience compression during use, operable to convert capacitance changes in the at least one capacitive sensing element into data for sending out over a communications link.

10. The orthopaedic bearing of any preceding claim, wherein the at least one capacitive sensing element is connected to at least one lead, which at least one lead follows an undulating path.

11. The orthopaedic bearing of claim 10, wherein the leads undulate at approximately 60°.

12. The orthopaedic bearing of any preceding claim, further comprising one or more locating legs adapted to locate the at least one capacitive sensing element correctly within the orthopaedic bearing.

13. The orthopaedic bearing of claim 12, wherein the one or more locating legs project from the corners of a module comprising the at least one capacitive sensing element.

14. The orthopaedic bearing of claim 13, wherein the module is substantially rectangular in shape and the one or more locating legs project at an angle that is substantially 45° to each adjacent side.

15. The orthopaedic bearing of any of claims 12 to 14, wherein the one or more locating legs have a bulbous distal end.

16. The orthopaedic bearing of any of claims 12 to 15, wherein the one or more locating legs are angled.

17. An orthopaedic bearing of any preceding claim, in which the bearing material is deformable under pressure according to a known characteristic.

18. An orthopaedic bearing of any preceding claim, in which the deformation of the deformable portion comprises compression thereof.

19. An orthopaedic bearing of any preceding claim, in which the deformation of the deformable portion comprises tension thereof.

20. The orthopaedic bearing of any preceding claim, wherein the flexible bearing material is a polymer material.

21. The orthopaedic bearing of any preceding claim, wherein the at least one capacitive sensing element comprises a pair of parallel overlapping plates.

22. The orthopaedic bearing of claim 21, comprising a plurality of capacitive sensing elements formed by a grid of overlapping conductors, said overlapping conductors comprising a first plurality of parallel and spaced apart conductors at a first orientation and a second plurality of parallel and spaced apart conductors at a second orientation, wherein a capacitive sensing element is formed at an overlapping junction between each one of the first and second plurality of parallel and spaced apart conductors.

23. The orthopaedic bearing of any of claim 22, wherein the first orientation is orthogonal to the second orientation.

24. The orthopaedic bearing of any of claims 21 to 23 inclusive, wherein compression of the orthopaedic bearing causes the parallel plates to move towards each other and tension of the orthopaedic bearing causes the parallel plates to move away from one another.

25. The orthopaedic bearing of any preceding claim, comprising at least one capacitive sensing element arranged within the bearing material, and operable to measure a change in capacitance resultant from any change in distance between a metallic bone insert and the capacitive sensing element within the orthopaedic bearing during use.

26. The orthopaedic bearing of any preceding claim, wherein the orthopaedic bearing is used with a tibial tray including a peg, and the orthopaedic bearing further comprises capacitive sensing elements arranged to measure a change in capacitance resultant from any deformation of the bearing on the peg during use.

27. The orthopaedic bearing of any preceding claim, wherein the orthopaedic bearing is used with a tibial tray and comprises a post that articulates with a femoral cam, and the orthopaedic bearing further comprises capacitive sensing elements arranged to measure a change in capacitance resultant from any compression or tension of the bearing caused by the articulating femoral cam during use.

28. The orthopaedic bearing of any preceding claim wherein the at least one capacitive sensing element comprises a plurality of capacitive sensing elements arranged within the at least one deformable portion of the orthopaedic bearing, wherein the plurality of capacitive sensing elements are positioned either in a plane parallel to the bottom of the bearing in use, or in a plane perpendicular to the bottom of the bearing in use, or a combination of either plane orientations.

29. The orthopaedic bearing of any preceding claim, wherein the at least one capacitive sensing element is formed from two adjacent co-planar plates operable to measure capacitance as a function of a proximity of the adjacent plates to a bone insert impinging on the orthopaedic bearing.

30. An orthopaedic implant comprising the orthopaedic bearing of any preceding claim.

31. A method of assessing an orthopaedic implant, comprising:
providing at least one orthopaedic bearing according to any of claims 1 to 29; and
determining, with the at least one orthopaedic bearing, at least one parameter of the orthopaedic implant during use.

32. The method of claim 31 further comprising using a combined mathematical and Finite Element model, run on a computer during post-processing, to translate a measured change in capacitance into information indicative of any one or more of: deformation of the orthopaedic bearing; forces acting upon the orthopaedic bearing; or wear of the orthopaedic bearing.

33. The method of claim 31 or 32, wherein the combined mathematical and Finite Element model uses calibration data together with the measured change in capacitance to estimate any one or more of: compression of a capacitive sensing element layer; a force that caused the compression; and/or a Centre Of Pressure of the force that caused the compression.
